(19) United States
(12) Patent Application Publication
(10) Pub. No.: US 2023/0316230 A1
(43) Pub. Date: Oct. 5, 2023

(54) DEVICE AND METHOD FOR PERSONALITY ASSESSMENT BASED ON NATURAL LANGUAGE PROCESSING

(71) Applicant: KOREA UNIVERSITY RESEARCH AND BUSINESS FOUNDATION, Seoul (KR)

(72) Inventors: Kee-Hong CHOI, Seoul (KR); Joon Yeon CHOEH, Seoul (KR); Seowon YOON, Seoul (KR)

(73) Assignee: KOREA UNIVERSITY RESEARCH AND BUSINESS FOUNDATION, Seoul (KR)

(21) Appl. No.: 18/127,166

(22) Filed: Mar. 28, 2023

(30) Foreign Application Priority Data
Mar. 30, 2022 (KR) 10-2022-0039465

(51) Int. Cl. G06Q 10/1053; G06F 40/40; G06F 40/205

(52) U.S. Cl. CPC G06Q 10/1053; G06F 40/205; G06F 40/40

(57) ABSTRACT

Disclosed is a natural language processing (NLP)-based personality assessment device. The personality assessment device includes a questionnaire providing unit configured to transmit, to a terminal, a message that includes a plurality of interview questions and at least one directive sentence and to receive, from the terminal, a first response to each of the plurality of interview questions and a second response to the at least one directive sentence; a preprocessing unit configured to preprocess at least one of the first response and the second response; and a personality prediction unit configured to predict personality of a user of the terminal using a preprocessing result by the preprocessing unit.

### CROSS-REFERENCE TO RELATED APPLICATIONS

This application claims the benefit under 35 USC § 119(a) of Korean Pat. Application No. 10-2022-0039465 filed on Mar. 30, 2022 in the Korean Intellectual Property Office, the entire disclosure of which is incorporated herein by reference for all purposes.

### BACKGROUND

### 1. Field

At least one example embodiment relates to a personality assessment method and, more particularly, to a device and method for automated personality assessment based on natural language processing.

### 2. Description of Related Art

In the field of psychological assessment that assesses an individual’s personality and/or mental health level, a self-report test is widely used. The self-report test exhibits some advantages, but has some inherent issues. That is, such self-report assessment is simple and highly serviceable, but face validity is too high, which may make it easy for an examinee to distort or bias a response. Response distortion and bias are more difficult to solve in job interviews or crime scenes.

Therefore, the present invention proposes a natural language processing (NLP)-based personality test device and method that may overcome limitations found in the self-report test, such as self-reported personality and distorted and biased responses. In detail, the present invention aims to develop a model (or an algorithm) for analyzing a subjective response to a question that may reveal personality and to predict personality by asking a unified question to all people.

### SUMMARY

A technical subject of at least one example embodiment is to provide a device and method for personality test based on natural language processing (NLP).

According to an aspect of an example embodiment, there is provided an NLP-based personality assessment device including a questionnaire providing unit configured to transmit, to a terminal, a message that includes a plurality of interview questions and at least one directive sentence and to receive, from the terminal, a first response to each of the plurality of interview questions and a second response to the at least one directive sentence; a preprocessing unit configured to preprocess at least one of the first response and the second response; and a personality prediction unit configured to predict personality of a user of the terminal using a preprocessing result by the preprocessing unit.

According to a personality test device and method according to an example embodiment, it is possible to overcome limitations found in the conventional self-report personality assessment using an NLP-based algorithm. Through an example embodiment, an examinee may show free speech and various unrestricted responses and may reveal personality or mental health condition closer to real life. Here, the examinee’s quality of response or extreme response tendency may also be included in analysis.

Also, to overcome limitations found in the existing language-based personality model of a bottom-up approach based on bigdata only, questions capable of revealing personality were developed based on scientifically verified psychological personality theory and provided to all the examinees. Therefore, even with a small sample, input noise is minimized such that personality traits may be revealed. The algorithm was developed based on high-quality data collected through direct interviews by researchers in the field of psychology instead of using simple data, such as social network service (SNS) data.

The present invention is expected to be applied in various fields. Most of all, the present invention may be applied in human resource management, such as job interviews at each company, medical institutions, and counseling centers. Also, the present invention may be applied even in the field, such as criminal assessment and schools. In particular, in the case of personnel assessment and personality assessment related to criminals, it is possible to distort and hide responses due to limitations found in the existing self-report test. Therefore, there is a lot of room to employ the assessment technique disclosed herein.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 illustrates a personality assessment system according to an example embodiment; and

FIG. 2 is a functional block diagram of a server of FIG. 1.

### DETAILED DESCRIPTION

Disclosed hereinafter are exemplary embodiments of the present invention. Particular structural or functional descriptions provided for the embodiments hereafter are intended merely to describe embodiments according to the concept of the present invention. The embodiments are not limited as to a particular embodiment.

Terms such as “first” and “second” may be used to describe various parts or elements, but the parts or elements should not be limited by the terms. The terms may be used to distinguish one element from another element. For instance, a first element may be designated as a second element, and vice versa, while not departing from the extent of rights according to the concepts of the present invention.

Unless otherwise clearly stated, when one element is described, for example, as being “connected” or “coupled” to another element, the elements should be construed as being directly or indirectly linked (i.e., there may be an intermediate element between the elements). Similar interpretation should apply to such relational terms as “between”, “neighboring,” and “adjacent to.”

Terms used herein are used to describe a particular exemplary embodiment and should not be intended to limit the present invention. Unless otherwise clearly stated, a singular term denotes and includes a plurality. Terms such as “including” and “having” also should not limit the present invention to the features, numbers, steps, operations, subparts and elements, and combinations thereof, as described; others may exist, be added or modified. Existence and addition as to one or more of features, numbers, steps, etc. should not be precluded.

Unless otherwise clearly stated, all of the terms used herein, including scientific or technical terms, have meanings which are ordinarily understood by a person skilled in the art. Terms, which are found and defined in an ordinary dictionary, should be interpreted in accordance with their usage in the art. Unless otherwise clearly defined herein, the terms are not interpreted in an ideal or overly formal manner.

Example embodiments of the present invention are described with reference to the accompanying drawings. However, the scope of the claims is not limited to or restricted by the example embodiments. Like reference numerals proposed in the respective drawings refer to like elements.

Hereinafter, example embodiments will be described with reference to the accompanying drawings. However, the scope of the patent application is not limited to or restricted by the example embodiments. Like reference numerals used herein refer to like elements throughout.

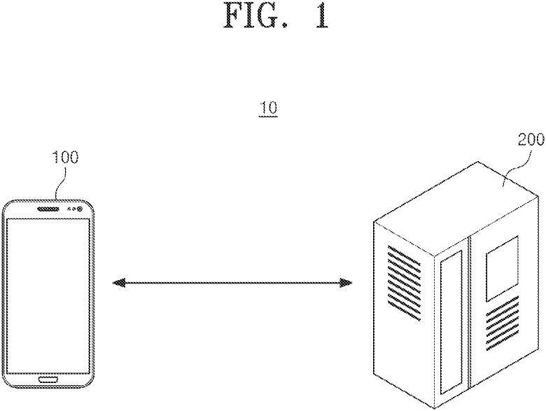

FIG. 1 illustrates a personality assessment system according to an example embodiment.

Referring to FIG. 1, a personality assessment system 10 may also be referred to as a personality measurement system and the like, and may include a terminal 100 and a server 200. The terminal 100 and the server 200 may exchange data through a predetermined wired and wireless communication network. Also, the terminal 100 and/or the server 200 may be implemented as a computing device that includes at least a processor and/or a memory. The computing device may include, for example, a mobile phone, a smartphone, a tablet personal computer (PC), a PC, a head mounted device (HMD), and a server.

The terminal 100 may receive a message from the server 200 through communication with the server 200 and may transmit a response thereto to the server 200 and, in this manner, may transmit data that is used as basic data for personality assessment or personality measurement to the server 200.

In detail, the terminal 100 may receive a plurality of interview questions from the server 200 and may transmit (subjective) responses thereto to the server 200. The plurality of interview questions may be questions for assessing personality traits of a user or an examinee. Each single interview question may be sequentially received or the plurality of interview questions may be received together.

Each of the plurality of questions may be text data or speech data. That is, each of the plurality of questions may include text data and may be displayed on a display device provided to the terminal 100. Each of the plurality of questions may include speech data. In this case, each of the plurality of questions may be output as a speech signal through a speech output device provided to the terminal 100. Depending on example embodiments, each of the plurality of questions may include text data and speech data. In this case, each of the plurality of questions may be displayed on the display device and, at the same time, may be output as a speech signal through the speech output device.

The terminal 100 may receive a response to each of the plurality of questions from the user or the examinee through a predetermined input interface and may transmit the received response to the server 200. Here, the response may be text data or speech data. However, a data form of each of the plurality of questions and a data form of the response may not match. That is, although each of the plurality of questions is in the form of text data, the response may be in the form of speech data. Although each of the plurality of questions is in the form of speech data, the response may be in the form of text data.

Also, the terminal 100 may also receive at least one directive sentence from the server 200. The at least one directive sentence may be received together with the plurality of questions or may be received separately from the plurality of questions. The directive sentence may also be text data or speech data. That is, the at least one directive sentence may include text data and may be displayed on the display device provided to the terminal 100. The at least one directive sentence may include speech data. In this case, the at least one directive sentence may be output as a speech signal through the speech output device provided to the terminal 100. Depending on example embodiments, the at least one directive sentence may include text data and speech data. In this case, the at least one directive sentence may be displayed on the display device and, at the same time, may be output as a speech signal through the speech output device.

The terminal 100 may receive a response corresponding to a directive sentence from the user or the examinee through a predetermined input interface and may transmit the received response to the server 200. Here, the response may include text data or speech data. However, a data form of the at least one directive sentence and a data form of the response may not match. That is, although each of the at least one directive sentence is in the form of text data, the response may be in the form of speech data. Although each of the at least one directive sentence is in the form of speech data, the response may be in the form of text data. The response to the at least one directive sentence may be simultaneously transmitted with the responses respectively corresponding to the plurality of questions, or may be transmitted separately from the responses respectively corresponding to the plurality of questions.

Also, although FIG. 1 illustrates only a single terminal, the right scope of the present invention is not limited thereto. That is, depending on example embodiments, the personality assessment system 10 may include a plurality of terminals.

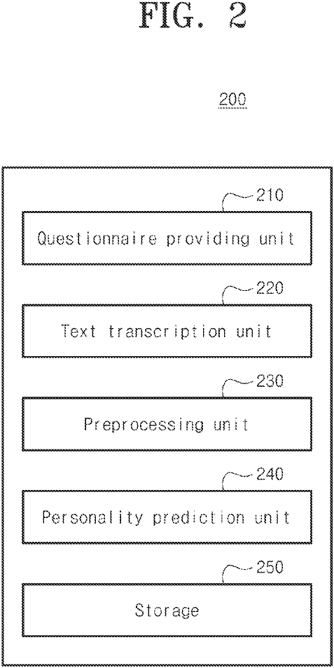

The server 200 may predict (or determine) personality of the user or the examinee based on the response of the user or the examinee received from the terminal 100 and may output a predicted (or determined) result. A detailed operation of the server 200 is described with reference to FIG. 2.

FIG. 2 is a functional block diagram of the server 200 of FIG. 1.

Referring to FIG. 2, the server 200 may also be referred to as a personality assessment device, a personality measurement device, a personality assessment server, a personality measurement server, and the like, and may include at least one of a questionnaire providing unit 210, a text transcription unit 220, a preprocessing unit 230, and a personality prediction unit 240. Depending on example embodiments, the server 200 may further include a storage 250.

The questionnaire providing unit 210 may transmit, to the terminal 100, a message that includes a plurality of interview questions and/or at least one directive sentence. Each of the plurality of interview questions may refer to a question for predicting personality of a user or an examinee. The number of interview questions may include n interview questions. Here, n denotes a natural number of 2 or more, for example, 18.

Examples of the interview question are as follows:
How do you want to spend your time for your routine daily hours?
To what extent do you tend to achieve the standard or the goal you have set for yourself?
How close do you want to be in relationships with people around you?
When you have a task to be completed within a set deadline, how do you usually handle it?
Talk about any negative feelings or thoughts you have.

Each of the plurality of interview questions is a question that requires a subjective response. The plurality of questions provided from the questionnaire providing unit 210 may be stored in the storage 250, and a (subjective) response to each of the plurality of questions received by the questionnaire providing unit 210 may be stored in the storage 250.

Examples of the at least one directive sentence transmitted from the questionnaire providing unit 210 are as follows:
List k adjectives that you think describe yourself well (here, k denotes a natural number of 2 or more, for example, 6).
Freely describe your personality including strengths and weaknesses (in 300 characters).

As described above, the at least one directive sentence may include a request for describing k (e.g., 6) adjectives that the user or the examinee thinks describe herself or himself well and/or a request for free description related to the personality. With respect to the at least one directive sentence provided from the questionnaire providing unit 210, the user or the examinee may transmit k (e.g., 6) adjectives that are thought to reveal the user or the examinee well in the form of text data or speech data through the terminal 100. The at least one directive sentence provided from the questionnaire providing unit 210 may be stored in the storage 250 and a response to the at least one directive sentence received by the questionnaire providing unit 210 may be stored in the storage 250.

The text transcription unit 220 may transcribe the response received from the terminal 100 to text data. That is, when a response in the form of speech data is present with respect to a subjective response to each of the plurality of questions received from the terminal 100 and/or a response to at least one directive sentence, the text transcription unit 220 may transcribe the response in the form of speech data to a response in the form of text data. Depending on example embodiments, the response from the user or the examinee may be in the form of text data. In this case, the text transcription unit 220 may be understood as a component that is deleted from the server 200.

The preprocessing unit 230 may perform a preprocessing operation on the response received by the questionnaire providing unit 210, the response received by the questionnaire providing unit 210 and stored in the storage 250, or the response transcribed to the form of text data by the text transcription unit 220. The preprocessing operation may include at least one of a segmentation (tokenization) operation, a word extraction operation, and a word selection operation. To this end, the preprocessing unit 230 may include at least one of a segmentation unit configured to perform the segmentation operation, a word extractor configured to perform the word extraction operation, and a word selector configured to perform the word selection operation.

The segmentation unit of the preprocessing unit 230 may perform the segmentation operation for the response (including at least a portion of the subjective response to each of the plurality of interview questions and the response to the at least one directive sentence). To perform the segmentation operation, at least one of the existing widely known methods may be used.

The word extractor of the preprocessing unit 230 may extract at least some words only from among words included in the response (including at least a portion of the subjective response to each of the plurality of interview questions and the response to the at least one directive sentence). The extracted words include at least an adjective, a verb, and a noun. To this end, the word extractor may determine part of speech (POS) for each of the segmented words, may extract words corresponding to the at least adjective, verb, and noun from among the words, and may exclude the remaining words. The word extractor may perform POS tagging to extract a word. POS tagging may be performed using at least one of the widely known methods. Examples of a POS tagging method may include a pointwise prediction method of individually predicting POS of each word using a classifier such as a support vector machine (SVM), a method of using a probability model such as an HMM or a conditional random field (CRF), and a method of using a neural network-based model, such as RNN, LSTM, and BERT. Depending on example embodiments, POS tagging may be simultaneously performed with the segmentation operation.

The word selector of the preprocessing unit 230 may select at least some words only from among words extracted by the word extractor using an LDA method and may exclude an unselected word. To this end, the word selector may extract a plurality of topics by applying the LDA method to the selected words. Each of the extracted plurality of topics and personality may have different correlation. Therefore, words included in a topic having a low correlation with the personality (or only the topic having the low correlation with the personality) may be excluded.

The correlation between the topic and the personality may be determined using various methods. Initially, the correlation (or a correlation level) may be measured by comparing a predetermined word list and keywords included in each topic. Words having a high (or determined to be high) correlation with the personality are included in the word list. Also, a weight of each of the words included in the word list may be computed according to a corresponding correlation level. In detail, for example, the correlation level may be computed as a weighted sum for keywords included in a topic. A topic having a correlation level of a predetermined threshold or less (or less than the predetermined threshold) or 0 may be determined as a topic irrelevant to the personality (or a topic less relevant to the personality) and words included in the topic determined as the topic irrelevant to the personality (or the topic less relevant to the personality) (or words included only in the topic) may be excluded. Here, if the correlation level is 0, it indicates a case in which none of the keywords included in the topic is included in the word list.

Then, selection information of an administrator (or an expert) for the extracted topics may be received and a topic that does not correspond to the received selection information may be determined as the topic irrelevant to the personality. To this end, the word selector of the preprocessing unit 230 may output the extracted topics through a predetermined output device (e.g., a display device) and may receive information on whether the topic is associated with the personality) from the administrator (or the expert). Therefore, words included in the topic that is determined as the topic irrelevant to the personality (or words included in the topic only) may be excluded.

As another example, a model configured to output a correlation level with the personality may be used for each of the extracted topics. To this end, a correlation decision model configured to receive keywords included in a topic and output a correlation (or correlation level) with the personality may be used. The correlation decision model may be generated by training a predetermined neural network model using training data that includes keywords of a topic and correlation information (e.g., correlation level) thereon. Words included in a topic having the output of the correlation decision model less than or equal to (or less than) a predetermined threshold or 0 may be excluded (or words included in the topic only). Depending on example embodiments, the correlation decision model may also output information on whether the topic has a correlation with the personality. In this case, words included in the topic that is determined to have no correlation (or words included in the topic only) may be excluded.

The personality prediction unit 240 may determine personality of the user (or examinee) based on a preprocessing result. The preprocessing result may represent a plurality of words that are finally selected. The personality prediction unit 240 may predict (or determine) personality of the user by embedding the plurality of words and then inputting the same to the personality prediction model (personality decision model).

To understand not the context of a word but meaning of a sentence after preprocessing, an embedding method showing the best performance among the existing Korean pretrained models, such as a transformer-based pretrained language model (KoBERT, etc.), within algorithms may be applied. Through this, text processing data received from the user is transcribed to numbers. For the personality prediction model, personality prediction points are computed by applying a parameter that is computed by applying a KoBERT-based finetuned model. A parameter computed by applying an algorithm that is trained using a technique showing the best performance among typical machine learning techniques (e.g., Boruta, Recursive Feature Elimination with Cross Validation (RFECV), Logistic Regression using Statsmodels, Random Forest, H2O, and L1 Regularization) with a deep learning method is applied for parameter extraction.

Through this, the conversion into personality points of each user is performed. In the case of an algorithm, for example, in a case in which the user uses words “friend” and “friends” (or in a case in which the user uses a word group similar to the word “friend”), points are measured with a weight for each extraversion. The word “friends” has a higher weight than that of the word “friend” and works to predict higher extroversion. On the contrary, word “alone” has a negative weight on extraversion and works to lower extraversion points. Word “depression” or “sadness” works to increase negative affectivity points and decrease emotional stability points. A linguistic response of each user may be provided with points for each of top five adaptive factors (extroversion (or extroversion-introversion), agreeableness, conscientiousness, openness, emotional stability), points for top five maladaptive factors (detachment, egocentrism, disinhibition (or attention deficit disorder), perceptual idiosyncrasy (or psychoticism), negative affectivity), and points for three to four subfactors for each upper factors, such that a personality profile may be computed.

That is, the personality prediction model may be generated by training a neural network or a language model (e.g., BERT or KoBERT as a pretrained language model) using predetermined training data. The training data may represent response data of the user in the form of text data and personality points corresponding thereto. The personality points may include points for each of the top five adaptive factors (which may include points for each subfactor) and or points for each of the top five maladaptive factors (which may include points for each subfactor).

Among the top adaptive factors, the extraversion refers to the tendency to gain vitality in relationships with people, expand interpersonal relationships, and exert influence on the surrounding, the agreeableness refers to the tendency to value smooth relationships, actively empathize with others, and act generously and trustfully, the conscientiousness refers to the tendency to value order and system and strive to achieve social customs and standards, the openness refers to the tendency to be curious about one’s inner self and the world and pursue new and diverse experience, and the emotional stability refers to the tendency to accept and express emotions in a healthy and mature way without suppressing or fearing the emotions.

Among the top maladaptive factors, the detachment refers to the tendency to avoid emotional exchange with people due to difficulty in experiencing positive emotions and low desire for interpersonal relationships, the egocentrism refers to the tendency to value one’s own interest and satisfaction and be insensitive to needs or emotions of others, the disinhibition refers to the tendency to have difficulty in controlling impulse to be suitable for context and situation and focusing attention on important stimuli, the psychoticism refers to the tendency to have difficulty in objectively perceiving the real world due to lack of flexibility in thinking and immersion in one’s own world, and the negative affectivity refers to the tendency to be easily aroused and overwhelmed by trivial stimuli due to frequency experience of negative emotions.

Among subfactors of the extraversion, vitality refers to the tendency to enjoy life without a lot of energy and enthusiasm, trust refers to the tendency to like new encounters and easily approach people and establish close relationships, assertiveness refers to the tendency to lead others due to strong self-assertion, and introversion refers to the tendency to prefer to live independently rather than to socialize with people.

Among subfactors of the agreeableness, trust refers to the tendency to value faith and loyalty in interpersonal relationships and behave transparently and honestly, generosity refers to the tendency to behave generously and moderately in interpersonal relationships and maintain amical relationships, and altruism refers to the tendency to emphasize with thoughts and emotions of others and actively consider and help others.

Among subfactors of the conscientiousness, orderly refers to the tendency to like rules and systems and to think and behave in an orderly manner, persistency refers to the tendency to make diligent and steady efforts to complete goals, and perfectionism refers to the tendency to aim for a high-level goal and achieve the same.

Among subfactors of the openness, experience openness refers to the exploratory tendency to like to directly experience new and diverse activities, intellectual openness refers to the tendency to have strong intellectual curiosity and enjoy philosophical and abstract thinking and reasoning, and aesthetic openness refers to the tendency to be sensitive art and beauty and enjoy rich and aesthetic activities.

Among subfactors of the emotional stability, emotion awareness refers to the tendency to pay attention to emotion one experiences and accurately and specifically understand the emotion, emotion acceptance refers to the tendency to naturally accept the emotion one experiences without distorting or suppressing the emotion, emotion expression refers to the tendency to naturally express emotion without being afraid of or having difficulty in revealing one’s emotions.

Among subfactors of the detachment, anhedonia refers to less experience positive emotions, such as joy and pleasure, suspiciousness refers to the tendency to be wary of people without trusting people due to fear of being unfairly harmed by others, and isolation refers to the tendency to have no interest or desire in interpersonal relationships and to keep distance without interacting with people.

Among subfactors of the egocentrism, narcissism refers to the tendency to believe that one is superior to others, has no flaws, and should be treated specially, histrionic refers to the tendency to exaggeratedly express oneself to gain attention and recognition from others, manipulativeness refers to the tendency to deceive others or behave according to one’s will to meet one’s own interests, and callousness refers to the tendency to behave harshly and callously due to not being able to empathize with situations and feelings of others.

Among subfactors of the disinhibition, impulsivity refers to the tendency to behave spontaneously without controlling one’s desire according to a situation, obsessiveness refers to the tendency to have difficulty in switching attention due to fixation on unimportant things, and distractibility refers to the tendency to be easily distracted and to find it difficult to focus on a single goal.

Among subfactors of the psychoticism, eccentricity refers to the tendency to think or behave in a peculiar and unconventional way unlike ordinary people, untunedness refers to the tendency to be unable to distinguish the inner world from reality and experience the world in a distorted way, and rigidity refers to the tendency to stubbornly think due to lack of flexibility and difficulty in tolerating ambiguous situations.

Among subfactors of the negative affectivity, anxiousness refers to the tendency to be chronically overly tense, vigilant, and worried, inferiority refers to the tendency to belittle and criticize oneself through chronical negative evaluation of oneself, and dependency refers to the tendency to rely entirely on others to cope with unstable emotions.

The storage 250 may store a program, an operating system (OS), and the like for performing a personality prediction operation by the server 200. Also, the storage 250 may store the plurality of interview questions and at least one directive sentence provided from the questionnaire providing unit 210, a subjective response to each of the plurality of interview questions and a subjective response to the at least one directive sentence received by the questionnaire providing unit 210, a result of text transcription by the text transcription unit 220, software for performing a text transcription operation, ad/or a speech recognition model, a preprocessing result by the preprocessing unit 230, data transitorily or non-transitorily generated during a preprocessing operation, a result of personality prediction by the personality prediction unit 240, data transitorily or non-transitorily generated during a personality prediction operation by the personality prediction unit 240, and a personality prediction model for personality prediction (i.e., trained model).

The aforementioned method for obtaining a vulnerable transaction sequence in a smart contract according to example embodiments may be implemented in a form of a program executable by a computer apparatus. Here, the program may include, alone or in combination, a program instruction, a data file, and a data structure. The program may be specially designed to implement the aforementioned method for obtaining a vulnerable transaction sequence in a smart contract or may be implemented using various types of functions or definitions known to those skilled in the computer software art and thereby available. Also, here, the computer apparatus may be implemented by including a processor or a memory that enables a function of the program and, if necessary, may further include a communication apparatus.

The program for implementing the aforementioned method for obtaining a vulnerable transaction sequence in a smart contract may be recorded in computer-readable record media. The media may include, for example, a semiconductor storage device such as an SSD, ROM, RAM, and a flash memory, magnetic disk storage media such as a hard disk and a floppy disk, optical record media such as disc storage media, a CD, and a DVD, magneto optical record media such as a floptical disk, and at least one type of physical device capable of storing a specific program executed according to a call of a computer such as a magnetic tape.

Although some example embodiments of an apparatus and method for obtaining a vulnerable transaction sequence in a program are described, the apparatus and method for obtaining a vulnerable transaction sequence in a program are not limited to the aforementioned example embodiments. Various apparatuses or methods implementable in such a manner that one of ordinary skill in the art makes modifications and alterations based on the aforementioned example embodiments may be an example of the aforementioned apparatus and method for obtaining a vulnerable transaction sequence in a program. For example, although the aforementioned techniques are performed in order different from that of the described methods and/or components such as the described system, architecture, device, or circuit may be connected or combined to be different form the above-described methods, or may be replaced or supplemented by other components or their equivalents, it still may be an example embodiment of the apparatus and method for obtaining a vulnerable transaction sequence in a program.

The device described above can be implemented as hardware elements, software elements, and/or a combination of hardware elements and software elements. For example, the device and elements described with reference to the embodiments above can be implemented by using one or more general-purpose computer or designated computer, examples of which include a processor, a controller, an ALU (arithmetic logic unit), a digital signal processor, a microcomputer, an FPGA (field programmable gate array), a PLU (programmable logic unit), a microprocessor, and any other device capable of executing and responding to instructions. A processing device can be used to execute an operating system (OS) and one or more software applications that operate on the said operating system. Also, the processing device can access, store, manipulate, process, and generate data in response to the execution of software. Although there are instances in which the description refers to a single processing device for the sake of easier understanding, it should be obvious to the person having ordinary skill in the relevant field of art that the processing device can include a multiple number of processing elements and/or multiple types of processing elements. In certain examples, a processing device can include a multiple number of processors or a single processor and a controller. Other processing configurations are also possible, such as parallel processors and the like.

The software can include a computer program, code, instructions, or a combination of one or more of the above and can configure a processing device or instruct a processing device in an independent or collective manner. The software and/or data can be tangibly embodied permanently or temporarily as a certain type of machine, component, physical equipment, virtual equipment, computer storage medium or device, or a transmitted signal wave, to be interpreted by a processing device or to provide instructions or data to a processing device. The software can be distributed over a computer system that is connected via a network, to be stored or executed in a distributed manner. The software and data can be stored in one or more computer-readable recorded medium.

A method according to an embodiment of the invention can be implemented in the form of program instructions that may be performed using various computer means and can be recorded in a computer-readable medium. Such a computer-readable medium can include program instructions, data files, data structures, etc., alone or in combination. The program instructions recorded on the medium can be designed and configured specifically for the present invention or can be a type of medium known to and used by the skilled person in the field of computer software. Examples of a computer-readable medium may include magnetic media such as hard disks, floppy disks, magnetic tapes, etc., optical media such as CD-ROM’s, DVD’s, etc., magneto-optical media such as floptical disks, etc., and hardware devices such as ROM, RAM, flash memory, etc., specially designed to store and execute program instructions. Examples of the program instructions may include not only machine language codes produced by a compiler but also high-level language codes that can be executed by a computer through the use of an interpreter, etc. The hardware mentioned above can be made to operate as one or more software modules that perform the actions of the embodiments of the invention and vice versa.

While the present invention is described above referencing a limited number of embodiments and drawings, those having ordinary skill in the relevant field of art would understand that various modifications and alterations can be derived from the descriptions set forth above. For example, similarly adequate results can be achieved even if the techniques described above are performed in an order different from that disclosed, and/or if the elements of the system, structure, device, circuit, etc., are coupled or combined in a form different from that disclosed or are replaced or substituted by other elements or equivalents. Therefore, various other implementations, various other embodiments, and equivalents of the invention disclosed in the claims are encompassed by the scope of claims set forth below.

Although the present invention is described with reference to the example embodiments illustrated in the drawings, it is provided as an example only and it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents. Therefore, other implementations, other example embodiments, and equivalents are within the scope of the following claims.

What is claimed is:

1. A personality assessment device comprising:
a questionnaire providing unit configured to transmit, to a terminal, a message that includes a plurality of interview questions and at least one directive sentence and to receive, from the terminal, a first response to each of the plurality of interview questions and a second response to the at least one directive sentence;
a preprocessing unit configured to preprocess at least one of the first response and the second response; and
a personality prediction unit configured to predict personality of a user of the terminal using a preprocessing result by the preprocessing unit.

2. The personality assessment device of claim 1, wherein each of the plurality of interview questions is a question for predicting the personality of the user, requiring a subjective response, and
the at least one directive sentence includes a directive sentence that requests a response to a plurality of adjectives that are thought to describe the user well and a directive sentence that requests a response to a strength and a weakness of the personality of the user.

3. The personality assessment device of claim 1, further comprising:
a text transcription unit configured to transcribe at least one of the first response in the form of speech data and the second response in the form of speech data to the form of text data.

4. The personality assessment device of claim 1, wherein the preprocessing unit is configured to perform a segmentation operation on at least one of the first response and the second response, to extract words corresponding to an adjective, a verb, and a noun from among words included in at least one of the first response and the second response, and to select some words from among the extracted words based on correlation with the personality.

5. The personality assessment device of claim 4, wherein the preprocessing unit is configured to extract a plurality of topics by applying a Latent Dirichlet Allocation (LDA) method to the extracted words and to select some words from among the extracted words based on correlation between each of the plurality of topics and the personality.

6. The personality assessment device of claim 5, wherein the preprocessing unit is configured to compute the correlation between each of the plurality of topics and the personality using a word list that includes a plurality of weighted words and to exclude a word included in a topic having the correlation of a predetermined threshold or less among the plurality of topics.

7. The personality assessment device of claim 5, wherein the preprocessing unit is configured to receive selection information on each of the plurality of topics and to exclude a word included in an unselected topic among the plurality of topics.

8. The personality assessment device of claim 5, wherein the preprocessing unit is configured to compute the correlation between each of the plurality of topics and the personality using a correlation decision model that outputs correlation with a topic and to exclude a word included in the topic having the correlation of the predetermined threshold or less among the plurality of topics.

9. The personality assessment device of claim 1, wherein the personality prediction unit is configured to predict the personality of the user by inputting a preprocessing result to a trained personality prediction model.

10. The personality assessment device of claim 9, wherein the personality prediction model is a transformer-based pretrained language model, and
the personality prediction model is configured to output prediction points for top five adaptive factors and top five maladaptive factors.
